(19) United States
(12) Patent Application Publication
(10) Pub. No.: US 2023/0372348 A1
(43) Pub. Date: Nov. 23, 2023

(54) LIPID FORMULATIONS OF TRIAZOLOQUINAZOLINONE COMPOUNDS

(71) Applicant: Gabather AB, Soedertaelje (SE)

(72) Inventors: Stefan REHNMARK, Tullinge (SE); Michael-Robin WITT, Soedertaelje (SE); Christine N. RYAN, Soedertaelje (SE)

(21) Appl. No.: 18/247,920

(22) PCT Filed: Oct. 6, 2021
(86) PCT No.: PCT/IB2021/000691

§ 371 Date: Apr. 5, 2023

Related U.S. Application Data
(60) Provisional application No. 63/087,957, filed on Oct. 6, 2020.

(51) Int. Cl. A61K 31/519; A61K 47/24; A61K 47/12; A61K 47/14; A61K 47/08; A61K 47/10; A61K 9/48

(52) U.S. Cl. CPC A61K 31/519; A61K 47/24; A61K 47/12; A61K 47/14; A61K 47/08; A61K 47/10; A61K 9/4825; A61K 9/4858

(57) ABSTRACT

The invention relates to pharmaceutical formulations of a triazoloquinazolinone compound of Formula (1) and Compound (1a) in particular, and pharmaceutically acceptable salts thereof in a substantially non-aqueous carrier. The substantially non-aqueous carrier used in a pharmaceutical formulation of the invention contains (i) a phospholipid composition, (ii) a non-ionic water dispersible surfactant, and (iii) optionally, an oleic acid; an ester of oleic acid, such as methyl oleate, ethyl oleate, and the like; or diethylene glycol monoethyl ether.

### CROSS-REFERENCE TO RELATED APPLICATIONS

This application claims priority to U.S. Provisional Application No. 63/087,957, filed on Oct. 6, 2020, the disclosure of which is incorporated by reference.

### TECHNICAL FIELD

This invention relates to pharmaceutical formulations and pharmaceutically acceptable salts of triazoloquinazolinone compounds.

### BACKGROUND OF THE INVENTION

The γ-aminobutyric acid_{A} (GABA_{A}) receptor complex is a pentameric assembly of several different protein subunits, which exist in multiple isoforms (α₁₋₆, β₁₋₃, γ₁₋₃, θ, π, ε, ρ₁₋₃ and δ). (Whiting, R J et al. “Molecular and functional diversity of the expanding GABAA receptor gene family” Annals of the NY Academy of Sciences 1999, April 30, pp 645-653.) The most abundant of these GABA_{A} receptors contain two α, two β and one γ subunits. Several ligands are known to allosterically modulate the GABA_{A} receptor, such as benzodiazepine (BDZ), barbiturates, ethanol and certain steroids. Historically, BDZ has attracted most attention and has as such been used clinically for treatment as anxiolytic, anticonvulsant, muscle relaxant, and sedative-hypnotic drugs. (Nutt, D J and Malizia, A L “New insights into the role of GABA_{A} receptors in psychiatric disorders”, Br. J. Psychiatry (2001), Vol. 179, pp 390-396.

Receptors with different subtype composition are associated with different physiological effects, e.g., α₁-containing receptors mediate sedation and anterograde amnesia, α₂-, and/or α₃-containing receptors are involved in anxiolytic activity, and α₅-containing receptors might be associated with cognition and memory. (Whiting, P J “GABAA receptor subtypes in the brain: a paradigm for CNS drug discovery”, Drug Discovery Today, Vol. 8, No. 10, May 2003, pp 445-450.) The α₁βₓγ₂, α₂βₓγ₂, α₃βₓγ₂ and α₅βₓγ₂ subtype assemblies are regard as the major benzodiazepine binding receptors, the benzodiazepine binding-site being located at the interface between the α and the γ subunit. BDZ receptor ligands are structurally different compounds, which bind to the (GABA_{A})/BDZ receptor complex. They display a broad pharmacological effect stretching from the full agonistic agents exhibiting anxiolytic, anticonvulsant, sedative-hypnotic and myorelaxant activities to the inverse agonistic agents, which displays anxiogenic, and pro-convulsant activities. In between, antagonistic agents that elicit no pharmacological effect are present.

Side effects characteristically produced by benzodiazepines include sedation, muscle relaxation, drug interaction effects, and dependence. (Bennett, D A “Pharmacology of the pyrazolo-type compounds: agonist, antagonist and inverse agonist actions”, Physiology & Behavior (1987) Vol. 41, pp. 241-245.) Among the wide variety of nonbenzodiazepine ligands, the most potent and perhaps best studied belong to the following classes: 2-arylpyrazoloquinolines, β-carbolines, pyridodiindoles, pyrimidin-5(6H)-ones, triazoloqunioxalines, cyclopyrrolones, and quinolines. U.S. Pat. No. 8,809,355.

One triazoloquinazolinone, the compound 9-benzyl-2-(4-methylphenyl)-2,6-dihydro[1,2,4]triazolo[4,3-c]quinazoline-3,5-dione (Compound (1a), shown below), is a very potent inhibitor of 3H-Flumazenil binding to the benzodiazepine site of the GABA_{A} receptor complex in rat membrane preparations. Compound (1a) is 10 times more potent than diazepam in displacing 3H-Flumazenil and is selective for the GABA_{A} receptor complex.

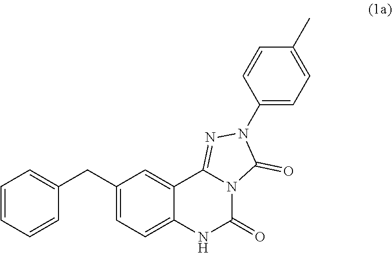 [SMILES: Cc1ccc(-n2nc3c4cc(Cc5ccccc5)ccc4[nH]c(=O)n3c2=O)cc1]

There is a need in the art to provide pharmaceutical formulations of triazoloquinazolinone compounds which allow for their therapeutic use with patients.

### SUMMARY OF THE INVENTION

The invention relates to pharmaceutical formulations of a triazoloquinazolinone compound of Formula (1) and Compound (1a) in particular, described below, and pharmaceutically acceptable salts thereof in a substantially non-aqueous carrier. The substantially non-aqueous carrier used in a pharmaceutical formulation of the invention contains (i) a phospholipid composition, (ii) a non-ionic water dispersible surfactant, and (iii) optionally, an oleic acid; an ester of oleic acid, such as methyl oleate, ethyl oleate, and the like; or diethylene glycol monoethyl ether.

### DETAILED DESCRIPTION OF THE INVENTION

The invention relates to pharmaceutical formulations of a triazoloquinazolinone compound of Formula (1) and Compound (1a) in particular, described below, and pharmaceutically acceptable salts thereof in a substantially non-aqueous carrier. A pharmaceutical formulation of the invention is typically an oral dosage form such as a tablet, a capsule, or other consumable formulation. Pharmaceutical formulations of the invention generally contain, for example, 0.005% to 99.9% by weight, 1% to 99% by weight, 5% to 95% by weight, 10% to 90% by weight, 20% to 80% by weight, or 30% to 70% by weight of a triazoloquinazolinone compound of Formula (1) or a pharmaceutically acceptable salt thereof with the remainder being the substantially non-aqueous carrier. A substantially non-aqueous carrier used in a pharmaceutical formulation of the invention comprises, consists essentially of or consists of (i) 35% to 75% by weight of a phospholipid composition, (ii) 25% to 55% by weight of a non-ionic water dispersible surfactant, and (iii) optionally, 5% to 15% by weight of oleic acid; an ester of oleic acid, such as methyl oleate, ethyl oleate, and the like; or diethylene glycol monoethyl ether. The triazoloquinazolinone compound of Formula (1) and Compound (1a) in particular, described below, and pharmaceutically acceptable salts is present in the pharmaceutical formulation in a therapeutically effective amount to treat an anxiolytic, anticonvulsant, sedative-hypnotic, myorelaxant, anxiogenic, somnolytic or convulsant condition, neurocognitive disorders or neurodegenerative condition, such as Alzheimer's disease or dementia, and other conditions described below, in a human patient in need thereof.

Triazoloquinazoline Compounds

Triazoloquinazolinones used in a pharmaceutical formulation of the invention are those of Formula (1) and their pharmaceutically acceptable salts.

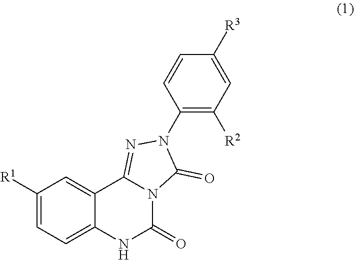

For a triazoloquinazolinone compound of Formula (1) R¹ is selected from the group consisting of halogen, alkyl having 1 to 2 carbon atoms, carboxyalkyl having 1 to 3 carbon atoms, phenyl-alkynyl-having 2 to 3 carbon atoms in the alkynyl chain, phenyl-alkenyl-having 2 to 3 carbon atoms in the alkenyl chain, phenyl-alkyl-having 1-3 carbon atoms in the alkyl chain and wherein the phenyl moiety may be further substituted by an oxygen-containing substituent (e.g. OH, OC₁-C₄ alkyl, OC(C₁-C₄ alkyl, etc.) or a sulphur-containing substituent (e.g. SH, SC₁-C₄ alkyl, etc.) in any position, pyridyl-alkyl-having 1 to 2 carbon atoms in the alkyl chain, and trifluoromethyl. When R¹ is halogen, it is a halogen selected from the group consisting of bromo, iodo, fluoro, and chloro. When R¹ is an alkyl having 1 to 2 carbon atoms, R¹ is a methyl or ethyl group. When R¹ is a carboxyalkyl, R¹ may be selected from the group consisting of carboxymethyl, carboxyethyl, and carboxypropyl. When R¹ is a phenylalkynyl, R¹ may be selected from the group consisting of phenylethynyl, phenyl-1-propynyl, and phenyl-2-propynyl. When R¹ is phenylalkenyl, R¹ may be selected from the group consisting of phenylethenyl, phenyl-1-propenyl, and phenyl-2-propenyl. When R¹ is phenylalkyl, R¹ may be selected from the group consisting of phenylmethyl, phenylethyl, phenylisopropyl, and phenylpropyl.

R² is selected from the group consisting of hydrogen and halogen. When R² is halogen, R² may be selected from the group consisting of bromo, iodo, fluoro, and chloro.

R³ is selected from the group consisting of hydrogen, halogen, and alkyl having 1 to 3 carbon atoms. When R³ is an alkyl having 1 to 3 carbon atoms, R³ is selected from the group consisting of methyl, ethyl propyl, and isopropyl. When R³ is halogen, R³ may be selected from the group consisting of bromo, iodo, fluoro, and chloro.

A particular triazoloquinazolinone compound of Formula (1) is 9-benzyl-2-(4-methylphenyl)-2,6-dihydro[1,2,4]triazolo[4,3-c]quinazoline-3,5-dione (Compound (1a), shown below) and its pharmaceutically acceptable salts.

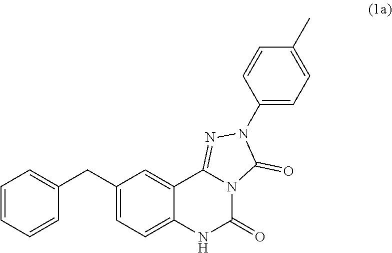 [SMILES: Cc1ccc(-n2nc3c4cc(Cc5ccccc5)ccc4[nH]c(=O)n3c2=O)cc1]

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula (1) or any of its intermediates. Illustrative inorganic acids which form suitable salts include hydrochloric, hydrobromic, sulfuric, and phosphoric acid and acid metal salts such as sodium monohydrogen orthophosphate and potassium hydrogen sulfate. Illustrative organic acids which form suitable salts include the mono-, di-, and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid, and other sulfonic acids such as methanesulfonic acid and 2-hydroxyethanesulfonic acid. Either the mono- or di-acid salts can be formed. In general, the acid addition salts of these compounds are more soluble in water and various hydrophilic organic solvents, and generally demonstrate higher melting points in comparison to their free base forms. One skilled in the art will know the selection criteria for the appropriate salt. Other non-pharmaceutically acceptable salts, e.g., oxalates, may be used for example in the isolation of compounds of Formula (1) for laboratory use, or for subsequent conversion to a pharmaceutically acceptable acid addition salt.

A “pharmaceutically acceptable basic addition salt” is any non-toxic organic or inorganic base addition salt of the acid compounds represented by Formula (1) or any of its intermediates. Illustrative inorganic bases which form suitable salts include lithium, sodium, potassium, calcium, magnesium, or barium hydroxides. Illustrative organic bases which form suitable salts include aliphatic, alicyclic, or aromatic organic amines such as methylamine, trimethyl amine and picoline or ammonia. The selection of the appropriate salt may be important so that an ester functionality, if any, elsewhere in the molecule is not hydrolyzed. One skilled in the art will know the selection criteria for the appropriate salt.

The term “stereoisomers” is a general term for all isomers of the individual molecules that differ only in the orientation of their atoms in space. It includes mirror image isomers (enantiomers), geometric (cis/trans) isomers, and isomers of compounds with more than one chiral center that are not mirror images of one another (diastereomers).

When a triazoloquinazolinone compound of Formula (1) contains one or more chiral centers, the compound may exist in, and be isolated as, enantiomeric or diastereomeric forms, or as a racemic mixture. The compounds include any possible enantiomers, diastereomers, racemates, or mixtures thereof, of a compound of Formula (1). The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

Certain triazoloquinazolinone compounds of Formula (1) may exist as geometrical isomers, for example E and Z isomers of alkenes. The invention includes any geometrical isomer of a compound of Formula (1). The invention also encompasses tautomers of the compounds of Formula (1).

Substantially Non-Aqueous Carriers

A “substantially non-aqueous” carrier has no water, or only an amount of water that is small enough to be, in practical terms, essentially non-deleterious to performance or properties of the pharmaceutical formulation. Typically, the carrier comprises zero (0) to less than about 5% by weight water or zero (0) to less than about 3% by weight water. It will be understood that some other ingredients used in a pharmaceutical formulation of the invention can themselves bind small amounts of water on or within their molecules or supramolecular structures; such bound water if present does not affect the “substantially non-aqueous” character of the carrier.

A substantially non-aqueous carrier used in a pharmaceutical formulation of the invention contains (i) 35% to 75% by weight of a phospholipid composition, (ii) 20% to 65% by weight of a non-ionic water dispersible surfactant, and (iii) optionally, 5% to 15% by weight of oleic acid; an ester of oleic acid, such as methyl oleate, ethyl oleate, and the like; or diethylene glycol monoethyl ether, where the amount of (i), (ii) and (iii) present combined are 100% by weight of the substantially non-aqueous carrier. A substantially non-aqueous carrier may contain (i) 50% to 75% by weight of a phospholipid composition, (ii) 20% to 50% by weight of a non-ionic water dispersible surfactant, and (iii) optionally, 5% to 15% by weight of oleic acid; an ester of oleic acid, such as methyl oleate, ethyl oleate, and the like; or diethylene glycol monoethyl ether; (i) 35% to 50% by weight of a phospholipid composition, (ii) 50% to 65% by weight of a non-ionic water dispersible surfactant, and (iii) optionally, 5% to 15% by weight of oleic acid; an ester of oleic acid, such as methyl oleate, ethyl oleate, and the like; or diethylene glycol monoethyl ether; (i) 35% to 50% or 40% by weight of a phospholipid composition and (ii) 50% to 65% or 60% by weight of a non-ionic water dispersible surfactant; (i) 35% to 50% by weight of a phospholipid composition, (ii) 35% to 55% by weight of a non-ionic water dispersible surfactant, and (iii) optionally, 5% to 15% by weight of oleic acid; an ester of oleic acid, such as methyl oleate, ethyl oleate, and the like; or diethylene glycol monoethyl ether; or (i) 40% by weight of a phospholipid composition, (ii) 50% by weight of a non-ionic water dispersible surfactant, and (iii) 10% by weight of oleic acid; an ester of oleic acid, such as methyl oleate, ethyl oleate, and the like; or diethylene glycol monoethyl ether.

Phospholipid Composition in the Non-Aqueous Carrier

A phospholipid composition, component (i) of the substantially non-aqueous carrier, contains a phospholipid and a solubilizing agent and other optional ingredients. The phospholipid may be a single phospholipid or a mixture of phospholipids. The solubilizing agent, likewise, may be a single solubilizing agent or a mixture of solubilizing agents. The phospholipid composition may contain at least 50% by weight of a phospholipid and about 20 to about 40% by weight of the solubilizing agent based on the total weight of the phospholipid composition or may contain at least 60% by weight of a phospholipid and about 20 to about 35% by weight of the solubilizing agent based on the total weight of the phospholipid composition. The total weight percent of the phospholipid(s), solubilizing agent(s) and optional ingredients, when present, in the phospholipid composition add to a total of 100 weight percent.

Any pharmaceutically acceptable phospholipid or mixture of phospholipids can be used. In general, such phospholipids are phosphoric acid esters that yield on hydrolysis phosphoric acid, fatty acid(s), an alcohol and a nitrogenous base. Pharmaceutically acceptable phospholipids include without limitation phosphatidylcholines (PCs), phosphatidylserines (PSs), phosphatidylethanolamines (PEs), phosphatidic acids (PAs), lysophosphatidylcholines (also known as phosphatidylinositols) (PIs), phosphatidylglycerols (PGs), and cardiolipins (CLs). A phosphatidylcholine may be a soybean phosphatidylcholine, an egg phosphatidylcholine, a synthetic phosphatidylcholine, a hydrogenated phosphatidylcholine or a mixture thereof. In one embodiment the composition comprises phosphatidylcholine, derived for example from natural lecithin. Any source of lecithin can be used, including animal sources such as egg yolk (an egg phosphatidylcholine), but plant sources are generally preferred. Soy is a particularly rich source of lecithin that can provide phosphatidylcholine (a soybean phosphatidylcholine) for use in the present invention. Accordingly, the phospholipid used may be a phosphatidylcholine derived from soy lecithin. In one embodiment the phosphatidylcholine is a soybean phosphatidylcholine, an egg phosphatidylcholine, a synthetic phosphatidylcholine, or a hydrogenated phosphatidylcholine; or is a soybean phosphatidylcholine or is an egg phosphatidylcholine; or is a soybean phosphatidylcholine; or is an egg phosphatidylcholine. The phospholipid may be a mixture of a phosphatidylcholine and a lysophosphatidylcholine.

The solubilizing agent may be selected from those known in the art and depend to some extent on the triazoloquinazolinone compound of Formula (1) used and its desired concentration. Exemplary solubilizing agents are selected from a glycol, a glyceride, or a combination thereof. Suitable glycols include, for example, propylene glycol and polyethylene glycols (PEGs) having molecular weight of about 200 to about 1,000 g/mol. e.g., PEG 400, which has an average molecular weight of about 400 g/mol, PEG 600, which has an average molecular weight of about 570-630 g/mol, and PEG 800, which has an average molecular weight of about 800 g/mol. Suitable glyceride materials include, without limitation, medium to long chain mono-, di- and triglycerides. “Medium chain” glycerides have individual hydrocarbyl chains of more than about 6 and less than about 12 carbon atoms, including for example C₈ to C₁₀ chains. Glycerides having palmyl or caprylyl and capryl chains, e.g. caprylic/capric mono-, di- and triglycerides, are examples of “medium chain” glycerides. “Long chain” glycerides have individual hydrocarbyl chains of at least about 12, for example about 12 to about 18, carbon atoms, including for example lauryl, myristyl, cetyl, stearyl, oleyl, linoleyl and linolenyl chains. Medium to long chain hydrocarbyl groups in a glyceride can be saturated, mono- or polyunsaturated. When the solubilizing agent is a glyceride the glyceride selected from the group consisting of medium and long chain mono-, di- and triglycerides, and mixtures thereof. The mono-, di- or triglyceride may also be prepared from vegetable oils, such as for example sunflower oil, soybean oil, sesame oil, which are mixtures of fatty acids. The solubilizing agent may be dimethyl isosorbide, available as Arlasolve® DMI from Uniqema, New Castle, DE, USA.

The phospholipid composition may also contain small amounts of other optional ingredients, such as ethanol; antioxidants (such as ascorbyl palmitate, butyl hydroxyl toluene (BHT) or the like); vegetable fatty acids (such as soy fatty acids); vegetable fatty acid esters (such as ascobyl palmitate) or a pharmaceutically acceptable surfactant. These other optional ingredients may individually be present in amounts of about 0.1 to about 10 percent by weight or about 1 to about 7 percent by weight based on the total weight based on the total weight of the phospholipid composition. The combined amount of such other optional ingredients in a phospholipid composition is typically less than 15 percent by weight based on the total weight of the phospholipid composition.

One phospholipid composition which may be used in a pharmaceutical formulation of the invention comprises:
not less than 53% by weight phosphatidylcholine,
not more than 6% by weight lysophosphatidylcholine,
about 29% by weight medium chain triglycerides,
3-6% by weight ethanol,
about 3% by weight mono- and diglycerides from sunflower oil,
about 2% by weight oleic acid, and
about 0.2% by weight ascorbyl palmitate.

Such a phospholipid composition is commercially available from Lipoid GmbH, Ludwigshafen Germany under the PHOSAL® 53 MCT tradename.

Another phospholipid composition which may be used in a pharmaceutical formulation of the invention comprises:
not less than 50% by weight phosphatidylcholine,
not more than 6% by weight lysophosphatidylcholine,
about 35% by weight propylene glycol,
about 3% by weight mono- and diglycerides from sunflower oil,
about 2% by weight soy fatty acids,
about 2% by weight ethanol, and
about 0.2% by weight ascorbyl palmitate.
   Such a phospholipid composition is commercially available from Lipoid GmbH, Ludwigshafen Germany under the PHOSAL® 50 SG tradename.

Non-Ionic Water Dispersible Surfactant in the Non-Aqueous Carrier

A substantially non-aqueous carrier used in a pharmaceutical formulation of the invention contains as component (ii) 25% to 55% by weight of a non-ionic water dispersible surfactant. Component (ii) may be present in an amount of 30% to 45% by weight or about 30% by weight. A non-ionic water dispersible surfactant of component (ii) may be the same or different from the optional surfactant in the phospholipid component (i). Examples of liquid non-ionic surfactants with an HLB above 10 that may be used in a pharmaceutical formulation according to the invention include, but are not limited to, sorbitan derivatives such as Polysorbate 20, TWEEN® 20, TWEEN® 40 and TWEEN® 80, SYNPERONIC™ L44, and polyoxyl 10-oleyl ether, all available from Uniqema or Croda, and polyoxyethylene containing surfactants e.g. PEG-8 caprylic/capric glycerides (e.g. Labrasol® PEG-8 or Labrasol® ALF available from Gattefosse of Eschbach, Germany). Labrafil® M2125CS (corn oil PEG-6 esters), also available Gattefosse, may also be used. In a pharmaceutical formulation of the invention the non-ionic water dispersible surfactant preferably comprises caprylocaproyl polyoxyl-8 glycerides (PEG-8 caprylic/capric glycerides).

Optional Components in the Non-Aqueous Carrier

The substantially non-aqueous carrier system may contain an optional third component. Optional component (iii) in a non-aqueous carrier is oleic acid; an ester of oleic acid, such as methyl oleate, ethyl oleate, and the like; or diethylene glycol monoethyl ether. Transcutol® HP (diethylene glycol monoethyl ether), also available from Gattefosse, may be used. The optional third component may be Crodamol™ EO ethyl oleate available from Croda. The optional third component may also be Super Refined™, oleic acid, available from Croda.

In an embodiment the invention relates to a pharmaceutical formulation comprising, consisting essentially of or consisting of a therapeutically effective amount of triazoloquinazolinone compound or a pharmaceutically acceptable salt thereof in a substantially non-aqueous carrier,
the triazoloquinazolinone compound is a compound of formula (1):

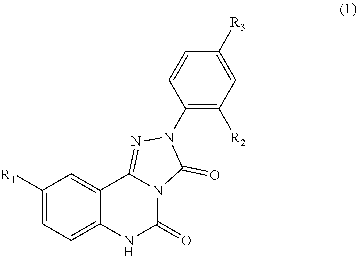

wherein
R₁ is selected from the group consisting of halogen, alkyl having 1 to 2 carbon atoms, carboxyalkyl having 1 to 3 carbon atoms, phenyl-alkynyl-having 2 to 3 carbon atoms in the alkynyl chain, phenyl-alkenyl-having 2 to 3 carbon atoms in the alkenyl chain, phenyl-alkyl-having 1-3 carbon atoms in the alkyl chain and wherein the phenyl moiety may be further substituted by an oxygen-containing substituent or a sulphur-containing substituent in any position, pyridyl-alkyl-having 1 to 2 carbon atoms in the alkyl chain, and trifluoromethyl,
R² is selected from the group consisting of hydrogen and halogen, and
R³ is selected from the group consisting of hydrogen, halogen and alkyl having 1 to 3 carbon atoms; and
   the substantially non-aqueous carrier comprises, consists essentially of or consists of:
(i) 35% to 75% by weight, or 50% to 75% by weight, or 35% to 50% by weight, or 40% by weight of a phospholipid composition,
(ii) 25% to 65% by weight, or 20% to 50% by weight, or 50% to 65% by weight, or 35% to 55% by weight, or 60% by weight, or 50% by weight of a non-ionic water dispersible surfactant, and
(iii) optionally, 5% to 15% by weight or 10% by weight of oleic acid, an oleic acid ester, or diethylene glycol monoethyl ether.
   In an embodiment of this pharmaceutical formulation the phospholipid composition comprises, consists essentially of or consists of at least 50% by weight of a phospholipid or a mixture of phospholipids; about 20 to about 40% by weight of a solubilizing agent or a mixture of solubilizing agents based on the total weight of the phospholipid composition; and optionally up to 15% by weight of an ingredient selected from the group consisting of ethanol, an antioxidant, a vegetable fatty acid, a vegetable fatty acid ester, or a pharmaceutically acceptable surfactant or mixtures thereof based on the total weight of the phospholipid composition. In another embodiment of this pharmaceutical formulation the phospholipid composition comprises, consists essentially of or consists of at least 60% by weight of a phospholipid or a mixture of phospholipids; about 20 to about 35% by weight of the solubilizing agent or a mixture of solubilizing agents based on the total weight of the phospholipid composition; and optionally up to 15% by weight of an ingredient selected from the group consisting of ethanol, an antioxidant, a vegetable fatty acid, a vegetable fatty acid ester, or a pharmaceutically acceptable surfactant or mixtures thereof based on the total weight of the phospholipid composition.

In an embodiment the invention relates to a pharmaceutical formulation comprising, consisting essentially of or consisting of a therapeutically effective amount of triazoloquinazolinone compound or a pharmaceutically acceptable salt thereof in a substantially non-aqueous carrier,
the triazoloquinazolinone compound is a compound of formula (1a):

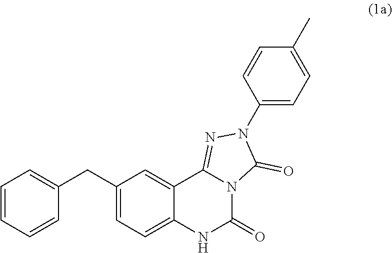 [SMILES: Cc1ccc(-n2nc3c4cc(Cc5ccccc5)ccc4[nH]c(=O)n3c2=O)cc1]

and
   the substantially non-aqueous carrier comprises, consists essentially of or consists of:
(i) 35% to 75% by weight, or 50% to 75% by weight, or 35% to 50% by weight, or 40% by weight of a phospholipid composition,
(ii) 25% to 65% by weight, or 20% to 50% by weight, or 50% to 65% by weight, or 35% to 55% by weight, or 60% by weight, or 50% by weight of a non-ionic water dispersible surfactant, and
(iii) optionally, 5% to 15% by weight or 10% by weight of oleic acid, an oleic acid ester, or diethylene glycol monoethyl ether.
   In an embodiment of this pharmaceutical formulation the phospholipid composition comprises, consists essentially of or consists of at least 50% by weight of a phospholipid or a mixture of phospholipids; about 20 to about 40% by weight of a solubilizing agent or a mixture of solubilizing agents based on the total weight of the phospholipid composition; and optionally up to 15% by weight of an ingredient selected from the group consisting of ethanol, an antioxidant, a vegetable fatty acid, a vegetable fatty acid ester, or a pharmaceutically acceptable surfactant or mixtures thereof based on the total weight of the phospholipid composition. In another embodiment of this pharmaceutical formulation the phospholipid composition comprises, consists essentially of or consists of at least 60% by weight of a phospholipid or a mixture of phospholipids; about 20 to about 35% by weight of the solubilizing agent or a mixture of solubilizing agents based on the total weight of the phospholipid composition; and optionally up to 15% by weight of an ingredient selected from the group consisting of ethanol, an antioxidant, a vegetable fatty acid, a vegetable fatty acid ester, or a pharmaceutically acceptable surfactant or mixtures thereof based on the total weight of the phospholipid composition.

Therapeutic Uses of the Pharmaceutical Formulations

The invention relates to pharmaceutical formulations containing a therapeutically effective amount of a triazoloquinazolinone compound of Formula (1), and pharmaceutically acceptable salts thereof in a substantially non-aqueous carrier, as described above, including the preferred embodiments. The invention relates to pharmaceutical formulations containing a therapeutically effective amount of 9-benzyl-2-(4-methylphenyl)-2,6-dihydro[1,2,4]triazolo[4,3-c]quinazoline-3,5-dione, or a pharmaceutically acceptable salt thereof in a substantially non-aqueous carrier, as described above, including the preferred embodiments. A pharmaceutical formulation of the invention may be used to treat Alzheimer's disease, cognitive disorders, memory deficits, schizophrenia, positive, negative and/or cognitive symptoms associated with schizophrenia, and cognitive deficits associated with Down syndrome, cognitive deficits associated with autism, cognitive deficits associated with neurofibromatosis type I, or cognitive deficits after stroke, stroke, various acute and chronic neurological disorders, chronic neurological disorders, bipolar disorders, sleep disorders, disorders of circadian rhythms, amyotrophic lateral sclerosis, dementia caused by AIDS, psychotic disorders, substance-induced psychotic disorder, anxiety disorders, generalized anxiety disorder, panic disorder, delusional disorder, obsessive compulsive disorders, acute stress disorder, drug addictions, movement disorders, Parkinson's disease, restless leg syndrome, cognition deficiency disorders, multi-infarct dementia, mood disorders, depression, neuropsychiatric conditions, psychosis, attention-deficit hyperactivity disorder, neuropathic pain, stroke, attentional disorders, eating disorders, anorexia nervosa, cachexia, weight loss, muscle atrophy, epilepsy, pain conditions, chronic pain, nociceptive pain, post-operative pain, osteoarthritis pain, rheumatoid arthritis pain, musculoskeletal pain, burn pain, ocular pain, pain due to inflammation, pain due to bone fracture, hyperalgesia, anaesthesia, neuropathic pain, herpes-related pain, HIV-related neuropathic pain, traumatic nerve injury, post-stroke pain, post-ischemia pain, fibromyalgia, chronic headache, migraine, tension-type headache, diabetic neuropathic pain, phantom limb pain, visceral pain, cutaneous pain, diabetes, itch, neurodegenerative diseases and substance abuse, in a human patient in need thereof. A pharmaceutical formulation of the invention may be used in treatments to achieve normalization of brain oscillations and brain synchronizations in pathological states in all bands of the electroencephalogram (EEG). A pharmaceutical formulation of the invention may be particularly used to treat Alzheimer's disease, cognitive disorders, depression or cognitive defects in depression.

A “therapeutically effective amount” of a triazoloquinazolinone compound of Formula (1), and pharmaceutically acceptable salts” is that which correlates to a therapeutic effect. A therapeutically effective amount, in terms of the amount of the compound given to a patient/subject (mg compound/patient) may for example, be about 0.025 mg to 250 mg, 0.025 mg to 100 mg, 0.025 mg to 50 mg, 0.025 mg to 25 mg, 0.025 mg to 10 mg, 0.05 mg to 10 mg, 0.025 mg to 5 mg, 0.05 mg to 5 mg, 0.025 mg to 2.5 mg, 0.05 mg to 2.5 mg, 0.05 mg to 2 mg, 0.5 mg, 1 mg, 1.5 mg, 2 mg or 2.5 mg. The actual amount required for treatment of any particular disease, disorder or condition for any particular patient may depend upon a variety of factors including, for example, the particular disease, disorder or condition being treated; the disease state being treated and its severity; the specific pharmaceutical composition employed; the age, body weight, general health, sex and diet of the patient; the mode of administration; the time of administration; the route of administration; and the rate of excretion; the duration of the treatment; any drugs used in combination or coincidental with the specific compound employed; and other such factors well known in the medical arts. These factors are discussed in Goodman and Gilman's “The Pharmacological Basis of Therapeutics”, Tenth Edition, A. Gilman, J. Hardman and L. Limbird, eds., McGraw-Hill Press, 155-173, 2001, which is incorporated herein by reference.

A pharmaceutical formulation of the invention may be an oral dosage formulation. For example, a therapeutically effective amount of a triazoloquinazolinone compound of Formula (1), such as 9-benzyl-2-(4-methylphenyl)-2,6-dihydro[1,2,4]triazolo[4,3-c]quinazoline-3,5-dione, or a pharmaceutically acceptable salt thereof, in a substantially non-aqueous carrier may be encapsulated or contained within a capsule, such as a hard or soft elastic gelation capsule. The components of the capsule shell may be selected from gelatin, glycerol, and purified water.

The invention also relates to methods of treating in a human patient in need thereof by administering a pharmaceutical formulation of the invention. The pharmaceutical composition may be administered orally. The invention relates to methods of treatment and the therapeutic use of a pharmaceutical formulation of the invention to treat Alzheimer's disease, cognitive disorders, memory deficits, schizophrenia, positive, negative and/or cognitive symptoms associated with schizophrenia, and cognitive deficits associated with Down syndrome, cognitive deficits associated with autism, cognitive deficits associated with neurofibromatosis type I, or cognitive deficits after stroke, stroke, various acute and chronic neurological disorders, chronic neurological disorders, bipolar disorders, sleep disorders, disorders of circadian rhythms, amyotrophic lateral sclerosis, dementia caused by AIDS, psychotic disorders, substance-induced psychotic disorder, anxiety disorders, generalized anxiety disorder, panic disorder, delusional disorder, obsessive compulsive disorders, acute stress disorder, drug addictions, movement disorders, Parkinson's disease, restless leg syndrome, cognition deficiency disorders, multi-infarct dementia, mood disorders, depression, neuropsychiatric conditions, psychosis, attention-deficit hyperactivity disorder, neuropathic pain, stroke, attentional disorders, eating disorders, anorexia nervosa, cachexia, weight loss, muscle atrophy, epilepsy, pain conditions, chronic pain, nociceptive pain, post-operative pain, osteoarthritis pain, rheumatoid arthritis pain, musculoskeletal pain, burn pain, ocular pain, pain due to inflammation, pain due to bone fracture, hyperalgesia, anaesthesia, neuropathic pain, herpes-related pain, HIV-related neuropathic pain, traumatic nerve injury, post-stroke pain, post-ischemia pain, fibromyalgia, chronic headache, migraine, tension-type headache, diabetic neuropathic pain, phantom limb pain, visceral pain, cutaneous pain, diabetes, itch, neurodegenerative diseases and substance abuse, in a human patient in need thereof. The invention relates to methods of treatment and the therapeutic use of a pharmaceutical formulation of the invention in treatments to achieve normalization of brain oscillations and brain synchronizations in pathological states in all bands of the electroencephalogram (EEG).

### EXAMPLES

The following materials were used to prepare the exemplary formulations used in the pharmacokinetic and other studies described in these examples.

PHOSAL® 53 MCT, available from Lipoid GmbH, Ludwigshafen, Germany, contains 53% phosphatidyl choline (PC) in MCT, alcohol, glycerol stearate, oleic acid, and ascorbyl palmitate.

Arlasolve® DMI, dimethyl isosorbide, available from Uniqema, New Castle, DE, USA.

Labrasol®, PEG-8 caprylic/capric glycerides available from Gattefosse, Eschbach, Germany.

Labrasol® ALF, PEG-8 caprylic/capric glycerides available from Gattefosse, Eschbach, Germany.

### Example 1—Formulation Preparation and Pharmacokinetic Evaluation

The general procedure for preparation of lipid formulations containing PHOSAL® 53 MCT was to prepare a stock solution of Compound (1a) (10 and/or 20 mg/g) in PHOSAL® 53 MCT. To this stock solution different excipients were added in different ratios according to what is described for each formulation, 1A-1L, as described below.

Stock solutions of a 10 mg/g Compound (1a) (in PHOSAL® 53 MCT (˜20 mg/g solution) were prepared as follows:
Weigh out the appropriate amount of Compound (1a) in a separate clear glass vial with airtight screw cap.
Add the appropriate amount of PHOSAL®53 MCT (weight/weight) to the glass vial with Compound (1a).
Place a magnetic stir bar in the glass vial.
Close the glass vial containing the Compound (1a)/PHOSAL® 53 MCT suspension and place the vial in an ultrasonic water bath at +50° C. to +65° C., sonicate and stir intermittently for about 1 hour.
Check the solution visually (light microscope at low magnification) to assure that it is completely clear and free of needle-like crystals.
If necessary, heat and stir (as above) until a clear solution is achieved.
Let the solution cool down to room temperature.
   The formulations tested are described below and summarized in Table 2 below.

Single dose pharmacokinetic evaluations of formulations F12-F22 were carried out using male Sprague Dawley rats using conditions as described in Table 1.

TABLE 1
Sprague Dawley Rats

| Study type: | Pharmacokinetic evaluation | |
|---|---|---|
| Species. strain and sex: | Male Sprague Dawley rat | |
| Duration of treatment: | 8 | hrs |
| Dosage (free base equivalent). | 10 | mg/kg PO |
| Dose volume (mL/kg): | 5 | PO |

| | |
|---|---|
| No. of animals/time point: | 3 |
| No. of animal (total): | 3 |
| Timepoints for sample | 0.25, 0.5, 1, 2, 3, 4, 5, 6 and 8 |
| collection (hrs) | |
| No. of test items: | 1 |
| Samples collected: | #Blood. EDTA coated collection tubes |
| | (Microvette, Sarstedt AB). |

Animal ID's were labelled by pen marks on tail and body weight was recorded for calculation of test item volume before administration. Blood was collected capillary from vena saphena from the rats in EDTA coated collection tubes and centrifuged for five minutes at 2000G. Plasma was transferred to pre-labelled Eppendorf tubes and immediately stored in −20° C. The pharmacokinetic analysis was performed using PK Solutions 2.0™, Noncompartmental Pharmacokinetic Data Analysis software. The pharmacokinetic parameters for the formulations are reported in Table 3 below.

Preparation of Formulation 1A

Compound (1a) was dissolved in PHOSAL® 53 MCT to prepare a stock solution of 10 mg/g, according to the procedure described above. To prepare formulation 1A the following excipients were added to the PHOSAL® 53 MCT stock solution to make a final concentration of 2 mg/g:
Compound (1a) (GT-002) PHOSAL® 53 MCT stock solution (10 mg/g): 3 g
PHOSAL® 53 MCT: 2 g
Labrasol® ALF: 5 g
Crodamol™ EO (ethyl oleate): 5 g
   The solution mixture (PHOSAL® 53 MCT 33 wt %, Labrasol® ALF 33 wt %, Crodamol™ EO 33 wt %) was heated at +50° C. to +65° C., intermittently stirred (magnetic bar) and sonicated for approximately 10 minutes.

Preparation of Formulation 1B

Compound (1a) was dissolved in PHOSAL® 53 MCT to prepare a stock solution of 10 mg/g, according to the procedure described above. To prepare formulation 1B the following excipients were added to the PHOSAL® 53 MCT stock solution to make a final concentration of 2 mg/g:
Compound (1a) PHOSAL® 53 MCT stock solution (10 mg/g): 0.8 g
PHOSAL® 53 MCT: 0.8 g
Arlasolve® DMI: 0.4 g
Super Refined™ oleic acid: 0.4 g
Labrasol® ALF: 1.6 g
   The solution mixture (40 wt % PHOSAL® 53 MCT, Labrasol® ALF 40 wt %, Arlasolve® DMI 10 wt %, Super Refined™ oleic acid 10 wt %) was heated at +50° C. to +65° C., intermittently stirred (magnetic bar) and sonicated for approximately 10 minutes.

Preparation of Formulation 1C

To prepare formulation 1C the following excipients were added to the PHOSAL® 53 MCT 20 mg/g stock solution to make a final concentration of 10 mg/g:
Compound (1a) PHOSAL® 53 MCT stock solution (10 mg/g): 0.5 g
Labrasol® ALF: 0.3 g
Arlasolve® DMI: 0.1 g
Super Refined™ oleic acid: 0.1 g
   The solution mixture (50 wt % PHOSAL®53 MCT, Labrasol® 30 wt %, Arlasolve® DMI 10 wt %, Super Refined™ oleic acid 10 wt %) was heated at +50° C. to +65° C., intermittently stirred (magnetic bar) and sonicated for approximately 10 minutes. Formulation 1C was not used in any PK or efficacy studies.

Preparation of Formulation 1D

To prepare formulation 1D the following excipients were added to the PHOSAL® 53 MCT 10 mg/g stock solution to make a final concentration of 2 mg/g:
Compound (1a) PHOSAL® 53 MCT stock solution (10 mg/g): 2 g
PHOSAL®53 MCT: 3 g
Labrasol® ALF: 4 g
Super Refined™ oleic acid: 1 g

The solution mixture (50 wt % PHOSAL® 53 MCT, Labrasol® ALF 40 wt %, Super Refined™ oleic acid 10 wt %) was heated at +50° C. to +65° C., intermittently stirred (magnetic bar) and sonicated for approximately 10 minutes.

Preparation of Formulation 1E

Formulation 1E only contains PHOSAL® 53 MCT with Compound (1a), which was used as a comparator to the pharmacokinetic properties of other formulations. Formulation 1E was prepared using the stock solution protocol above to making a 2 mg/g Compound (1a) PHOSAL® 53 MCT solution. The solution mixture (100% PHOSAL®53 MCT) was heated at +50° C. to +65° C., intermittently stirred (magnetic bar) and sonicated for approximately 10 minutes.

Preparation of Formulation 1F

To prepare formulation 1F the following excipient was added to the PHOSAL® 53 MCT 20 mg/g stock solution to make a final concentration of 10 mg/g:
PHOSAL® 53 MCT Compound (1a) stock solution (20 mg/g): 2 g
Polysorbate 20: 2 g
   The solution mixture (50% PHOSAL® 53 MCT, Polysorbate 20 50%) was heated at +50° C. to +65° C., intermittently stirred (magnetic bar) and sonicated for approximately 10 minutes.

Preparation of Formulation 1G

To prepare formulation 1G the following excipients were added to the PHOSAL® 53 MCT 10 mg/g stock solution to make a final concentration of 2 mg/g:
Compound (1a) PHOSAL® 53 MCT stock solution (10 mg/g): 1 g
PHOSAL® 53 MCT: 4.5 g
Labrasol® ALF: 3 g
Tween® 20 (polysorbate): 0.5 g
Super Refined™ oleic acid: 1 g
   The solution mixture (55 wt % PHOSAL® 53 MCT, Labrasol® ALF 30 wt %, Tween® 20 5 wt %, Super Refined™ oleic acid 10 wt %) was heated at +50° C. to +65° C., intermittently stirred (magnetic bar) and sonicated for approximately 10 minutes. Formulation 1G was not tested in the pharmacokinetic studies.

Preparation of Formulation 1H

To prepare the F18 formulation the following excipients were added to the PHOSAL® 53 MCT 10 mg/g stock solution to make a final concentration of 2 mg/g:
Compound (1a) PHOSAL® 53 MCT stock solution (10 mg/g): 2 g
Compound (1a) PHOSAL® 53 MCT: 5 g
Labrasol® ALF: 3 g
   The solution mixture (70% PHOSAL® 53 MCT, Labrasol® ALF 30%) was heated at +50° C. to +65° C., intermittently stirred (magnetic bar) and sonicated for approximately 10 minutes.

Preparation of Formulation 1I

To prepare formulation 1I the following excipients were added to the PHOSAL® 53 MCT 10 mg/g stock solution to make a final concentration of 2 mg/g:
Compound (1a) PHOSAL® 53 MCT stock solution (10 mg/g): 2 g
Labrasol® ALF: 8 g
   The solution mixture (PHOSAL® 53 MCT 20 wt %, Labrasol® ALF 80 wt %) was heated at +50° C. to +65° C., intermittently stirred (magnetic bar) and sonicated for approximately 10 minutes.

Preparation of Formulation 1J

To prepare formulation 1J the following excipients were added to the PHOSAL® 53 MCT 10 mg/g stock solution to make a final concentration of 2 mg/g:
Compound (1a) PHOSAL® 53 MCT stock solution (10 mg/g): 4 g
PHOSAL® 53 MCT: 4 g
Labrasol® ALF: 10 g
Transcutol® HP (diethylene glycol monoethyl ether): 2 g
   The solution mixture (PHOSAL® 53 MCT 40 wt %, Labrasol® ALF 50 wt %, Transcutol® 10 wt %) was heated at +50° C. to +65° C., intermittently stirred (magnetic bar) and sonicated for approximately 10 minutes.

Preparation of Formulation 1K

To prepare formulation 1K the following excipients were added to the PHOSAL® 53 MCT 10 mg/g stock solution to make a final concentration of 2 mg/g:
Compound (1a) PHOSAL® 53 MCT stock solution (10 mg/g): 6 g
Labrasol® ALF: 7.5 g
Transcutol® HP (diethylene glycol monoethyl ether): 1.5 g

The solution mixture (PHOSAL® 53 MCT 40 wt %, Labrasol® ALF 50 wt %, Transcutol® 10 wt %) was heated at +50° C. to +65° C., intermittently stirred (magnetic bar) and sonicated for approximately 10 minutes. This formulation was used in the following PK and efficacy studies. This formulation was reconstituted with water before dosing the rats, 4 ml dH₂0+9 ml 1K to make F.C. 2 mg/ml.

Preparation of Formulation 1L

To prepare formulation 1L the following excipients were added to the PHOSAL® 53 MCT 10 mg/g stock solution to make a final concentration of 2 mg/g:
Compound (1a) PHOSAL® 53 MCT stock solution (10 mg/g): 6 g
Labrasol® ALF: 9 g
   The solution mixture (PHOSAL® 53 MCT 40 wt %, Labrasol® ALF 60 wt %) was heated at +50° C. to +65° C., intermittently stirred (magnetic bar) and sonicated for approximately 10 minutes.

Summary of Formulation Composition and Pharmacokinetic Parameters

Table 2 shows the composition of formulations described above. Table 3 reports the pharmacokinetic parameters from the various rat PK studies. Cmax (nM) is the maximum concentration. AUC 0-24 (nM*h) is the area under the curve over 24 hours. Tmax (h) is the maximum time in hours. F % is bioavailability. Frel % is the relative bioavailability.

TABLE 2
Compositions of Lipid Formulations 1A-1L (wt %) with 10 mg Compound (1a).

| | PHOSAL ® | | | | | | | |
|---|---|---|---|---|---|---|---|---|
| | 53 | Labrasol ® | | Arlasove | Oleic | Crodamol ™ | Tween ® | Polysorbate |
| Formulation | MCT | ALF | Transcutol ® | DMI | Acid | EO | 20 | 20 |
| 1A | 33% | 33% | | | | 33% | | |
| Labarasol ALF | | 100% | | | | | | |
| 1B | 40% | 40% | | 10% | 10% | | | |
| 1C | 50% | 30% | | 10% | 10% | | | |
| 1D | 50% | 40% | | | 10% | | | |
| 1E | 100% | | | | | | | |
| 1F | 50% | | | | | | | 50% |
| (reconstituted | | | | | | | | |
| with water | | | | | | | | |
| 1:4) | | | | | | | | |
| 1G | 55% | 30% | | | 10% | | 5% | |
| 1H | 70% | 30% | | | | | | |
| 1I | 20% | 80% | | | | | | |
| 1J | 40% | 50% | 10% | | | | | |
| 1K | 40% | 50% | 10% | | | | | |
| (reconstituted | | | | | | | | |
| with water | | | | | | | | |
| 1:4) | | | | | | | | |
| 1L | 40% | 60% | | | | | | |

TABLE 3
Summary of Pharmacokinetic Results of Different Formulations

| Compound (1a) | Rat (10 mg/kg) | | | | |
|---|---|---|---|---|---|
| in formulations | Cmax | AUC 0-24 | Tmax | | |
| (avg. of n = 3) | (nM) | (nM*h) | (h) | F % | Frel % |
| Intravenously | | 1178 | | 100% | |
| (1 mg/kg) | | | | | |
| Labrasol ALF | 184 | 784 | 2.0 | 7% | 100% |
| (ref. formulation) | | | | | |
| 1A | 250 | 630 | 1.5 | 5% | 80% |
| 1B | 833 | 3224 | 2.0 | 27% | 411% |
| 1C | n/d | n/d | n/d | n/d | n/d |
| 1D | 1335 | 5957 | 3.3 | 51% | 760% |
| 1E | 688 | 3284 | 5.0 | 28% | 419% |
| 1F | 181 | 653 | 2.0 | 6% | 83% |
| 1G | n/d | n/d | n/d | n/d | n/d |
| 1H | 1827 | 8302 | 3.7 | 70% | 1059% |
| 1I | 270 | 730 | 1.7 | 6% | 93% |
| 1J | 1026 | 5630 | 4.3 | 48% | 718% |
| 1K | 52 | 218 | 2.0 | 2% | 28% |
| 1L | 792 | 3339 | 3.0 | 28% | 426% |

### Example 2—Pharmacokinetic Profile of Formulation 1H in Beagle Dogs

A Compound (1a) formulation 1H stock solution was prepared as described above. The formulation was then filled into hard gelatine capsules. The appropriate number of capsules required for administration was prepared on the administration day or on the day before the administration day. The capsules were stored in plastic boxes with dividers which separated the capsules for individual animals.

The Compound (1a) 1H formulation was administered orally by the gelatin capsules once daily for 4 weeks. 24 animals (12 males and 12 females) were divided into 4 groups {3 animals/sex/group):
Group 1: Control (vehicle)
Group 2: 0.1 mg/kg b.w./day
Group 3: 0.5 mg/kg b.w./day
Group 4: 2.0 mg/kg b.w./day

The amount of test item was adjusted to each animal's current body weight weekly. The control animals received the same number of capsules filled with the vehicle Formulation 1H (i.e., one capsule/animal/day). The administration was carried out approximately 2 hours before start of feeding. The PK results are shown in Table 4 below.

TABLE 4
Pharmacokinetic Results in Beagle Dogs

| | | | | | | | | |
|---|---|---|---|---|---|---|---|---|
| Cₘₐₓ^{#1} | Tₘₐₓ^{#1} | | Kₑₗ | AUC_{0-t last} | AUC_{0-∞} | AUC_{0-∞/dose} | | |
| [ng/mL] | [h] | t_{1/2} [h] | [1/h] | [ng * h/mL] | [ng * h/mL] | [h * kg * ng/mL/mg] | DPF | R |

Test day 1 (male)
Group 2: 0.1 mg/kg

| | | | | | | | | |
|---|---|---|---|---|---|---|---|---|
| 3.24 | 1.33 | 1.02 | 0.69 | 6.74 | 8.12 | 81.20 | n.a. | n.a. |

Group 3: 0.5 mg/kg

| | | | | | | | | |
|---|---|---|---|---|---|---|---|---|
| 39.10 | 1.33 | 0.76 | 0.93 | 79.97 | 78.11 | 156.22 | 2.28 | n.a. |

Group 4: 2.0 mg/kg

| | | | | | | | | |
|---|---|---|---|---|---|---|---|---|
| 267.33 | 1.67 | 0.84 | 0.83 | 637.72 | 640.18 | 320.09 | 4.73 | n.a. |

Test day 1 (female)
Group 2: 0.1 mg/kg

| | | | | | | | | |
|---|---|---|---|---|---|---|---|---|
| 4.79 | 1.00 | 0.97 | 0.74 | 7.46 | 8.97 | 89.73 | n.a. | n.a. |

Group 3: 0.5 mg/kg

| | | | | | | | | |
|---|---|---|---|---|---|---|---|---|
| 30.33 | 1.33 | 0.84 | 0.83 | 55.52 | 57.75 | 115.51 | 1.49 | n.a. |

Group 4: 2.0 mg/kg

| | | | | | | | | |
|---|---|---|---|---|---|---|---|---|
| 366.67 | 1.00 | 0.87 | 0.80 | 713.46 | 715.62 | 357.81 | 4.78 | n.a. |

Test day 28 (male)
Group 2: 0.1 mg/kg

| | | | | | | | | |
|---|---|---|---|---|---|---|---|---|
| 3.85 | 1.67 | 0.99 | 0.70 | 7.40 | 8.86 | 88.57 | n.a. | 1.10 |

Group 3: 0.5 mg/kg

| | | | | | | | | |
|---|---|---|---|---|---|---|---|---|
| 30.15 | 1.50 | 0.89 | 0.78 | 66.32 | 67.44 | 134.87 | 1.79 | 0.86 |

Group 4: 2.0 mg/kg

| | | | | | | | | |
|---|---|---|---|---|---|---|---|---|
| 331.33 | 2.00 | 0.94 | 0.74 | 791.92 | 797.13 | 398.57 | 5.35 | 1.24 |

Test day 28 (female)
Group 2: 0.1 mg/kg

| | | | | | | | | |
|---|---|---|---|---|---|---|---|---|
| 2.99 | 1.67 | 0.79 | 0.89 | 6.63 | 7.52 | 75.23 | n.a. | 0.89 |

Group 3: 0.5 mg/kg

| | | | | | | | | |
|---|---|---|---|---|---|---|---|---|
| 38.17 | 1.33 | 0.86 | 0.82 | 77.29 | 78.04 | 156.07 | 2.33 | 1.39 |

Group 4: 2.0 mg/kg

| | | | | | | | | |
|---|---|---|---|---|---|---|---|---|
| 306.33 | 1.33 | 0.89 | 0.78 | 637.91 | 640.66 | 320.33 | 4.81 | 0.89 |

#1: Values obtained from plasma analysis, all other values calculated by pharmacokinetic analysis
n.a.: Not applicable
Cₘₐₓ: Maximum concentration
Tₘₐₓ: Maximum time
t_{1/2}: Half-life
Kₑₗ: Elimination rate constant
AUC: Area under the curve
$DPF : Dose proportion factor = \frac{\begin{matrix}{AUC}_{0 - t last} ( x mg / kg ) / {AUC}_{0 - t last} \\ ( 0.1 mg / kg )\end{matrix}}{( x mg / kg ) / 0.1 mg / kg )}$ R: Accumulation factor = (AUC_{TD28 0-t last}/AUC_{TD1 0-t last})

### Example 3—Pharmacokinetic Profile of Formulation 1H in Healthy Male Subjects

Lipid formulations containing PHOSAL® 53 MCT delivered higher exposure in rat and dog when compared to other formulation (e.g., nanosuspension formulation and PEG formulations). Furthermore, the combination of PHOSAL® 53 MCT with Labrasol® ALF, with the highest concentration of PHOSAL® 53 MCT showed the highest plasma exposure, both in rat and dog. Formulation 1H containing 70% PHOSAL® 53 MCT and 30% Labrasol® ALF was selected for manufacturing of soft gelatin capsules for clinical trials with a drug formulation fill weight of 500 mg and a total capsule weight is 696 mg. Two dose strength (0.1 mg/subject and 2 mg/subject) and placebo were produced according to GMP for clinical testing. Tables 5-7 below show the composition of the drug product and placebo capsules.

TABLE 5
Composition of Compound (1a) Drug Product 0.05 mg capsules

| | Quantitative | |
|---|---|---|
| | amount per | |
| Component | capsule (mg) | Function |
| Compound (1a) | 0.050 | Active Ingredient |
| PHOSAL ® 53 MCT | 349.895 | Phospholipid Composition |
| Labrasol ® | 149.955 | Surfactant |
| Butyl hydroxyl toluene | 0.100 | Antioxidant |
| (BHT), EP | | |
| Gelatin | 109.006505 | Capsule shell |
| Glycerol | 71.313495 | Capsule shell |
| Purified water (MQ) | 15.680 | Capsule shell |
| Total capsule weight | 696.00 | |

TABLE 6
Composition of Compound (1a) Drug Product 1 mg capsules

| | Quantitative | |
|---|---|---|
| | amount per | |
| Component | capsule (mg) | Function |
| Compound (1a) | 1.000 | Active Ingredient |
| PHOSAL ® 53 MCT | 349.230 | Phospholipid Composition |
| Labrasol ® ALF | 149.670 | Surfactant |
| BHT, EP | 0.100 | Anti-oxidant |
| Gelatin | 109.006505 | Capsule shell |
| Glycerol | 71.313495 | Capsule shell |
| Purified water (MQ) | 15.680 | Capsule shell |
| Total capsule weight | 696.00 | |

TABLE 7
Composition of Placebo capsules

| | Quantitative | |
|---|---|---|
| | amount per | |
| Component | capsule (mg) | Function |
| PHOSAL ® 53 MCT | 349.930 | Phospholipid |
| Labrasol ® ALF | 149.970 | Surfactant |
| BHT, EP | 0.100 | Anti-oxidant |
| Gelatin | 109.006505 | Capsule shell |
| Glycerol | 71.313495 | Capsule shell |
| Water (MQ) | 15.680 | Capsule shell |
| Total capsule weight | 696.00 | |

The clinical development programme in healthy volunteers started with a double-blind, randomised, placebo controlled single ascending dose (SAD) study in males. Seven dose cohorts received increasing oral doses of Compound (1a) or placebo, dose range 0.05-2 mg. Each cohort consisted of 5-6 subjects receiving Compound (1a) and 2 subjects receiving placebo, with the primary objective to evaluate safety and tolerability, the secondary objective being evaluation of pharmacokinetics (PK).

A summary of PK results from the SAD study are shown below in Table 8.

TABLE 8
Summary of Pharmacokinetic Parameters for all Cohorts.
Dose Levels (mg)

| | 0.05 | 0.1 | 0.3 | 0.5 | 1 | 2 |
|---|---|---|---|---|---|---|
| Cₘₐₓ [ng/ml] | 0.61 (0.17) | 1.24 (0.230 | 3.99 (0.43) | 6 (1.35) | 15.99 (3.04) | 31.48 (9.27) |
| Tₘₐₓ [h]^{†} | 2 (0.7, 2.5) | 2 (1, 4) | 2.5 (2, 3) | 2 (1.5, 4) | 1.5 (1, 3) | 2 (1, 4) |
| AUC₇₂ | 6.77 (5.28) | 24.51 (10.3) | 100.71 | 80.51 | 296.87 | 621.89 |
| [h*ng/mL]* | | | (30.42) | (34.09) | (100.61) | (413.33) |
| AUC₀₋₂₄ | 5.61 (2.27) | 14.34 (4.25) | 55.52 (9.91) | 59.51 | 187.61 | 353.21 |
| [h*ng/mL] | | | | (18.56) | (48.45) | (147.72) |
| AUC_{inf} | 8.52 (5.76) | 28.2 (12.59) | 120.75 | 83.99 | 327.76 | 671.92 |
| [h*ng/mL] | | | (42.36) | (36.01) | (118.890 | (490.76) |
| T_{1/2} [h] | 13.47 (6.4) | 20.88 (9.21) | 25.72 (8.92) | 11.95 (4.62) | 18.79 (6.36) | 18.93 |
| | | | | | | (10.47) |
| C12 [ng/mL] | 0.21 (0.09) | 0.57 (0.2) | 2.2 (0.44) | 2.35 (0.93) | 7.53 (2.01) | 13.79 (5.91) |
| C24 [ng/mL] | 0.12 (0.1) | 0.39 (0.19) | 1.73 (0.53) | 1.22 (0.73 | 4.93 (1.91) | 9.18 (6.37) |

^{†}Tₘₐₓ is described by median (min, max).
*Dose level 2 mg has AUC₉₆ instead.
Cₘₐₓ: Maximum concentration
Tₘₐₓ: Maximum time
AUC: Area under the curve
T_{1/2}: Half-life
C12: Concentration at 12 hours
C24: Concentration at 24 hours

The Cₘₐₓ was dose proportional within the dose range tested in this study (0.05 mg-2 mg). The results obtained with an exploratory power model analysis did not fully support the assumption of dose proportionality for AUC_{inf} and AUC₇₂, but the study was probably lacking statistical power for formal testing of dose-proportionality with this approach. However, the use of unweighted linear regression analysis or ANOVA with a linear mixed model both supported dose proportionality of AUC_{inf} and AUC₇₂. Exposure to Compound (1a) appears to be close to dose proportional in the dose range of 0.05 to 2 mg, when the drug is administered orally as soft gelatin capsules to fasting, healthy male volunteers under standardized conditions.

The claimed invention is:

1. A pharmaceutical formulation comprising, consisting essentially of, or consisting of a therapeutically effective amount of triazoloquinazolinone compound or a pharmaceutically acceptable salt thereof in a substantially non-aqueous carrier
the triazoloquinazolinone compound is a compound of formula (1): 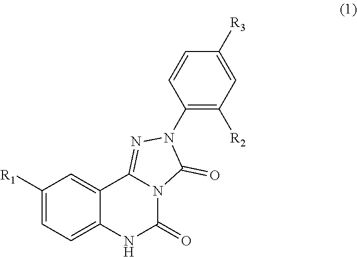
wherein
R¹ is selected from the group consisting of halogen, alkyl having 1 to 2 carbon atoms, carboxyalkyl having 1 to 3 carbon atoms, phenyl-alkynyl-having 2 to 3 carbon atoms in the alkynyl chain, phenyl-alkenyl-having 2 to 3 carbon atoms in the alkenyl chain, phenyl-alkyl-having 1-3 carbon atoms in the alkyl chain and wherein the phenyl moiety may be further substituted by an oxygen-containing substituent or a sulphur-containing substituent in any position, pyridyl-alkyl-having 1 to 2 carbon atoms in the alkyl chain, and trifluoromethyl,
R² is selected from the group consisting of hydrogen and halogen, and
R³ is selected from the group consisting of hydrogen, halogen and alkyl having 1 to 3 carbon atoms; and
the substantially non-aqueous carrier comprises:
(i) 35% to 75% by weight of a phospholipid composition,
(ii) 25% to 65% by weight of a non-ionic water dispersible surfactant, and
(iii) optionally, 5% to 15% by weight of oleic acid, and ester of oleic acid, or diethylene glycol monoethyl ether.

2. The pharmaceutical formulation of claim 1, wherein the phospholipid composition comprises a phospholipid and a solubilizing agent.

3. The pharmaceutical formulation of claim 1 or 2 wherein the phospholipid composition comprises (1) at least 50% by weight of a phospholipid or a mixture of phospholipids and about 20 to about 40% by weight of a solubilizing agent or a mixture of solubilizing agents based on the total weight of the phospholipid composition or comprises (2) at least 60% by weight of a phospholipid or a mixture of phospholipids and about 20 to about 35% by weight of the solubilizing agent or a mixture of solubilizing agents based on the total weight of the phospholipid composition.

4. The pharmaceutical formulation of any one of claims 1-3, wherein the phospholipid is a phosphatidylcholine derived from soy lecithin.

5. The pharmaceutical formulation of any one of claims 1-3, wherein solubilizing agent is selected from a glycol, a glyceride material, or a combination thereof.

6. The pharmaceutical formulation of claim 5, wherein the solubilizing agent comprises a glyceride material selected from the group consisting of medium and long chain mono-, di- and triglycerides, and mixtures thereof.

7. The pharmaceutical formulation of any one of claims 1-6, wherein the phospholipid composition comprises ethanol.

8. The pharmaceutical formulation of any one of claims 1-7, wherein the phospholipid composition further comprises a pharmaceutically acceptable surfactant.

9. The pharmaceutical formulation of any one of claims 1-8, wherein the phospholipid composition comprises:
not less than 53% by weight phosphatidylcholine based on the weight of the phospholipid composition,
not more than 6% by weight lysophosphatidylcholine based on the weight of the phospholipid composition,
about 29% by weight medium chain triglycerides based on the weight of the phospholipid composition,
3-6% by weight ethanol based on the weight of the phospholipid composition,
about 3% by weight mono- and diglycerides from sunflower oil based on the weight of the phospholipid composition,
about 2% by weight oleic acid based on the weight of the phospholipid composition, and
about 0.2% by weight ascorbyl palmitate based on the weight of the phospholipid composition.

10. The pharmaceutical formulation of any one of claims 1-9, wherein the phospholipid composition comprises:
not less than 50% by weight phosphatidylcholine based on the weight of the phospholipid composition,
not more than 6% by weight lysophosphatidylcholine based on the weight of the phospholipid composition,
about 35% by weight propylene glycol based on the weight of the phospholipid composition,
about 3% by weight mono- and diglycerides from sunflower oil based on the weight of the phospholipid composition,
about 2% by weight soy fatty acids based on the weight of the phospholipid composition,
about 2% by weight ethanol based on the weight of the phospholipid composition, and
about 0.2% by weight ascorbyl palmitate based on the weight of the phospholipid composition.

11. The pharmaceutical formulation of any one of claims 1-10, wherein the non-ionic water dispersible surfactant comprises caprylocaproyl polyoxyl-8 glycerides.

12. The pharmaceutical formulation of any one of claims 1-11, wherein the substantially non-aqueous carrier consists of:
(i) 35% to 75% by weight of a phospholipid composition containing
not less than 53% phosphatidylcholine based on the weight of the phospholipid composition,
not more than 6% lysophosphatidylcholine based on the weight of the phospholipid composition,
about 29% medium chain triglycerides based on the weight of the phospholipid composition,
3-6% ethanol based on the weight of the phospholipid composition,
about 3% mono- and diglycerides from sunflower oil based on the weight of the phospholipid composition,
about 2% oleic acid based on the weight of the phospholipid composition, and
about 0.2% ascorbyl palmitate based on the weight of the phospholipid composition; and
(ii) 25% to 55% by weight of a non-ionic water dispersible surfactant containing caprylocaproyl polyoxyl-8 glycerides;
(iii) optionally, 5% to 15% by weight of oleic acid or diethylene glycol monoethyl ether.

13. The pharmaceutical formulation of claim 12, wherein the substantially non-aqueous carrier consists of 70% of the phospholipid composition and 30% of the non-ionic water dispersible surfactant.

14. The pharmaceutical formulation of claim 12, wherein the non-aqueous drug carrier system consists of 40% of the phospholipid composition and 60% of the non-ionic water dispersible surfactant.

15. The pharmaceutical formulation of claim 12, wherein the non-aqueous drug carrier system consists of 40% of the phospholipid composition and 50% of the non-ionic water dispersible surfactant, and 10% diethylene glycol monoethyl ether.

16. The pharmaceutical formulation of claim 12, wherein the non-aqueous drug carrier system consists of 50% of the phospholipid composition and 40% of the non-ionic water dispersible surfactant, and 10% oleic acid.

17. The pharmaceutical formulation of any one of claims 1-16, wherein the triazoloquinazolinone compound is 9-benzyl-2-(4-methylphenyl)-2,6-dihydro[1,2,4]triazolo[4,3-c]quinazoline-3,5-dione.

18. The pharmaceutical formulation of claim 1, further comprising a capsule shell, suitable for oral administration, wherein the drug-carrier system is encapsulated.

19. The pharmaceutical formulation of claim 18, wherein the capsule shell is a hard or soft elastic gelatin capsule shell.

20. Use of a pharmaceutical formulation of any one of claims 1-19 to treat Alzheimer's disease, cognitive disorders, memory deficits, schizophrenia, positive, negative and/or cognitive symptoms associated with schizophrenia, and cognitive deficits associated with Down syndrome, cognitive deficits associated with autism, cognitive deficits associated with neurofibromatosis type I, or cognitive deficits after stroke, stroke, various acute and chronic neurological disorders, chronic neurological disorders, bipolar disorders, sleep disorders, disorders of circadian rhythms, amyotrophic lateral sclerosis, dementia caused by AIDS, psychotic disorders, substance-induced psychotic disorder, anxiety disorders, generalized anxiety disorder, panic disorder, delusional disorder, obsessive compulsive disorders, acute stress disorder, drug addictions, movement disorders, Parkinson's disease, restless leg syndrome, cognition deficiency disorders, multi-infarct dementia, mood disorders, depression, neuropsychiatric conditions, psychosis, attention-deficit hyperactivity disorder, neuropathic pain, stroke, attentional disorders, eating disorders, anorexia nervosa, cachexia, weight loss, muscle atrophy, epilepsy, pain conditions, chronic pain, nociceptive pain, post-operative pain, osteoarthritis pain, rheumatoid arthritis pain, musculoskeletal pain, burn pain, ocular pain, pain due to inflammation, pain due to bone fracture, hyperalgesia, anaesthesia, neuropathic pain, herpes-related pain, HIV-related neuropathic pain, traumatic nerve injury, post-stroke pain, post-ischemia pain, fibromyalgia, chronic headache, migraine, tension-type headache, diabetic neuropathic pain, phantom limb pain, visceral pain, cutaneous pain, diabetes, itch, neurodegenerative diseases and substance abuse or in treatments to achieve normalization of brain oscillations and brain synchronizations in pathological states in all bands of the electroencephalogram (EEG), in a human patient in need thereof.

21. A method of treating Alzheimer's disease, cognitive disorders, memory deficits, schizophrenia, positive, negative and/or cognitive symptoms associated with schizophrenia, and cognitive deficits associated with Down syndrome, cognitive deficits associated with autism, cognitive deficits associated with neurofibromatosis type I, or cognitive deficits after stroke, stroke, various acute and chronic neurological disorders, chronic neurological disorders, bipolar disorders, sleep disorders, disorders of circadian rhythms, amyotrophic lateral sclerosis, dementia caused by AIDS, psychotic disorders, substance-induced psychotic disorder, anxiety disorders, generalized anxiety disorder, panic disorder, delusional disorder, obsessive compulsive disorders, acute stress disorder, drug addictions, movement disorders, Parkinson's disease, restless leg syndrome, cognition deficiency disorders, multi-infarct dementia, mood disorders, depression, neuropsychiatric conditions, psychosis, attention-deficit hyperactivity disorder, neuropathic pain, stroke, attentional disorders, eating disorders, anorexia nervosa, cachexia, weight loss, muscle atrophy, epilepsy, pain conditions, chronic pain, nociceptive pain, post-operative pain, osteoarthritis pain, rheumatoid arthritis pain, musculoskeletal pain, burn pain, ocular pain, pain due to inflammation, pain due to bone fracture, hyperalgesia, anaesthesia, neuropathic pain, herpes-related pain, HIV-related neuropathic pain, traumatic nerve injury, post-stroke pain, post-ischemia pain, fibromyalgia, chronic headache, migraine, tension-type headache, diabetic neuropathic pain, phantom limb pain, visceral pain, cutaneous pain, diabetes, itch, neurodegenerative diseases and substance abuse or in treatments to achieve normalization of brain oscillations and brain synchronizations in pathological states in all bands of the electroencephalogram (EEG), comprising administering to the patient a pharmaceutical formulation of any one of claims 1-19.
